(19) United States
(12) Patent Application Publication
(10) Pub. No.: US 2015/0273132 A1
(43) Pub. Date: Oct. 1, 2015

(54) System and Method for Continuous Separation of Whole Blood

(71) Applicant: HAEMONETICS CORPORATION, Braintree, MA (US)

(72) Inventors: Michael Ragusa, Hingham, MA (US); Shiven Ruparel, Waltham, MA (US); Dominique Uhlmann, Abington, MA (US)

(21) Appl. No.: 14/440,245

(22) PCT Filed: Nov. 5, 2012
(86) PCT No.: PCT/US12/63574

§ 371 Date: May 1, 2015

(51) Int. Cl. A61M 1/36; B04B 11/02

(52) U.S. Cl. CPC A61M 1/3696; B04B 11/02

(57) ABSTRACT

An apparatus for separating whole blood includes a continuous flow separation device in which drawn whole blood is separated into a first and second blood component. The separation device has an inlet for introducing whole blood into the separation device, a first blood component outlet for withdrawing the first blood component, and a second blood component outlet for withdrawing the second blood component. The apparatus also includes a first blood component storage container connected to the first blood component outlet, and a first blood component pump to draw the first blood component from the separation device. Additionally, the apparatus can have second blood component storage container connected to the second blood component outlet, and a controller for controlling fluid flow through the apparatus. The controller monitors the total volume of whole blood drawn from the source, and stops a draw pump when the target whole blood volume is withdrawn.

### TECHNICAL FIELD

The present invention relates to systems and methods for blood processing, and more particularly to systems and methods for continuous separation of whole blood and collection of blood components.

### BACKGROUND ART

In prior art methods for collecting and separating whole blood, a technician places a needle into a vein in the donor's arm, causing whole blood to flow (e.g., by gravity) into a storage bag which may hold a quantity of anticoagulant solution to prevent the collected blood from clotting. After collecting a fixed volume of whole blood (e.g., approximately 400 ml+/−10%) from the donor, the technician removes the needle and the donor is free to leave. The technician then repeats the blood collection step for a number of donors with varying hematocrits (the ratio of the volume of packed red blood cells to the volume of whole blood—typically 38% to 60%). After collecting whole blood from each of the donors, the technician then transports the whole blood bags to a laboratory for processing.

Once in the laboratory, the technician places the whole blood bags into a large centrifuge which spins at a high rate of speed to separate the whole blood within the bags into its constituent components. The technician then removes the bags from the centrifuge (taking care not to re-mix the separated blood components) and transfers the bags to a device such as an expressor to remove plasma from the bag (e.g., leaving red blood cells remaining in the bag). After some additional processing, each of the individual components may then be stored separately.

As one may expect, prior art methods such as those described above are labor intensive, and require numerous manual manipulations. Additionally, because the whole blood must be transported to the lab, the whole blood must be stored for up to several hours prior to processing.

### SUMMARY OF THE EMBODIMENTS

In accordance with one embodiment of the present invention, an apparatus for separating whole blood includes an access device through which whole blood may be drawn from a source, a continuous flow separation device, a draw line, and a draw pump. The continuous flow separation device (e.g., including a continuous flow centrifuge bowl) is configured to separate whole blood into a first blood component and a second blood component. The continuous flow separation device may have (1) an inlet for introducing whole blood into the continuous flow separation device, (2) a first blood component outlet for withdrawing a first blood component, and (3) a second blood component outlet for withdrawing a second blood component. The draw line may fluidly connect the access device and the continuous flow separation device, and the draw pump may draw whole blood from the source through the access device and draw line and into the continuous flow separation device.

The apparatus may also include a first blood component storage container fluidly connected to the first blood component outlet, and a first blood component pump. The first blood component pump may draw the first blood component from the continuous flow separation device and into the first blood component storage container. In addition to the first blood component storage container, the apparatus can also include a final first blood component storage container fluidly connected to the first blood component outlet, and a second blood component storage container fluidly connected to the second blood component outlet. A controller may control fluid flow through the apparatus, and control the operation of the draw pump. The controller can also monitor a total volume of whole blood drawn from the source, and may stop the draw pump when the target whole blood volume (e.g., 400 mL or 450 mL) is withdrawn. The controller may determine the total volume of whole blood withdrawn based upon a number of revolutions of the draw pump.

In some embodiments, the first blood component pump may reintroduce the first blood component in the first blood component storage container into the continuous flow separation device. In such embodiments, the apparatus may also include a line sensor that (1) monitors fluid flowing out of the continuous flow separation device as the first blood component is reintroduced, and (2) outputs a signal representative of the fluid. The controller may receive the output signal and control the operation of the first blood component pump based, at least in part, upon the output signal. The separation device may have an optical sensor (located on the bowl) that monitors fluid within the continuous flow separation device. The controller may also control the operation of the first blood component pump based upon an output of the optical sensor.

In additional embodiments, the continuous flow separation device may allow simultaneous collection of the first and second blood components, and the source may be a whole blood storage container containing a volume of whole blood substantially equal to the target donor draw whole blood volume plus an appropriate amount of anticoagulant. The controller may control a speed of the continuous flow separation device based, at least in part, upon a hematocrit value of the drawn whole blood.

The first blood component storage container may collect pre-washed first blood component, and the first blood component pump can reintroduce the pre-washed first blood component within the first blood component collection container into the continuous flow separation device. The apparatus may also include an additive solution container containing additive solution, and an additive solution line fluidly connecting the additive solution container and the first blood component outlet of the continuous flow separation device. The first blood component pump may draw additive solution from the additive solution container through the additive solution line and into the continuous flow separation device to wash the first blood component within the continuous flow separation device (e.g., to reduce the protein concentration within the first blood component).

Additionally, the first blood component pump may be configured to draw the washed first blood component from the continuous flow separation device and into the final first blood component collection container. Alternatively, the apparatus may have a final first blood component pump configured to draw the washed first blood component from the continuous flow separation device and into the final first blood component collection container. The apparatus may also have a filter for filtering the washed first blood component.

In some embodiments, the source may be a donor and the apparatus may include an anticoagulant storage container, an anticoagulant pump for metering in anticoagulant at a fixed ratio in comparison to whole blood drawn from donor, and an anticoagulant line fluidly connected to the draw line for introducing anticoagulant into the drawn whole blood. Alternatively, the source may be a whole blood storage container containing anticoagulated whole blood.

In accordance with further embodiments, a method for separating whole blood and collecting blood components may include (1) drawing whole blood from a source, (2) introducing, using a draw pump, the whole blood into a continuous flow separation device, (3) separating, in the separation chamber, the whole blood into a first blood component and a second blood component (e.g., while whole blood is being drawn from the source), (4) extracting the first blood component from the separation chamber through the first blood component outlet, and (5) collecting the extracted first blood component within a first blood component storage container. The continuous flow separation device may have an inlet, a first blood component outlet, and a second blood component outlet. The whole blood may be introduced into the continuous flow separation device through the inlet, and may displace the second blood component from the continuous flow separation device via the second blood component outlet.

The method may also include collecting the displaced second blood component within a second blood component storage container while extracting the first blood component, and monitoring a total volume (based upon the number of revolutions of the draw pump) of whole blood drawn from the source to determine when a target whole blood volume is drawn. The method may stop (e.g., using a controller) the draw of whole blood from the source when the target whole blood volume is reached.

In some embodiments, the method may also include reintroducing the first blood component collected within the first blood component storage container into the continuous flow separation device to force additional second blood component out of the continuous flow separation device via the second blood component outlet. The method may then collect the additional second blood component within the second blood component storage container. While reintroducing the first blood component, a line sensor may monitor, the fluid flowing out of the continuous flow separation device and output a signal representative of the fluid. The method may stop the reintroduction of first blood component from the first blood component container when the line sensor detects cellular material.

The method may also include drawing additive solution from an additive solution container and into the continuous flow separation device. The additive solution may wash the reintroduced first blood component within the continuous flow separation device, and the method may then extract the washed first blood component from the continuous flow separation device and collect it within a final first blood component storage container. The method may use a first blood component pump to extract the first blood component from the continuous flow separation device, and use a final first blood component pump to extract the washed first blood component. Alternatively, the method may use the first blood component pump to extract the first blood component and the washed first blood component.

The method may then determine if additional first blood component remains within the first blood component storage container, and transfer the additional first blood component within the first blood component container to the continuous flow separation chamber. The method may also (1) draw additional additive solution from the additive solution container and into the continuous flow separation device to wash the additional first blood component within the continuous flow separation device, (2) extract the washed additional first blood component from the continuous flow separation device, and (3) collect the washed additional first blood component within the final first blood component container.

The source may be a whole blood storage container containing anticoagulated whole blood. Alternatively, the source may be a donor, and the method may include (1) connecting the donor to the access device prior to drawing whole blood, and (2) disconnecting the donor when the target volume of whole blood is withdrawn. The method can also draw anticoagulant from an anticoagulant storage container, and add the drawn anticoagulant to the drawn whole blood. The continuous flow separation device may include a continuous flow centrifugal bowl that includes the inlet, first blood component outlet, and second blood component outlet.

In accordance with additional embodiments, an apparatus for separating whole blood may include an access device through which whole blood is drawn from a source, and a continuous flow separation device in which the drawn whole blood is separated into a first blood component, a second blood component, and a third blood component. The continuous flow separation device may have an inlet for introducing whole blood into the continuous flow separation device, a first blood component outlet for withdrawing the first blood component, and a second blood component outlet for withdrawing the second blood component.

The apparatus may also include a draw line fluidly connecting the access device and the continuous flow separation device, and a draw pump configured to draw whole blood from the source through the access device and draw line and into the continuous flow separation device. To store the various blood components, the apparatus may also include a first blood component storage container fluidly connected to the first blood component outlet, a final first blood component storage container fluidly connected to the first blood component outlet, a second blood component storage container fluidly connected to the second blood component outlet, and a third blood component storage container fluidly connected to the second blood component outlet. A first blood component pump can draw the first blood component from the continuous flow separation device and into the first blood component storage container, and a second blood component pump may be fluidly connected to the second blood component container and the continuous flow separation device and may recirculate second blood component collected within the second blood component container to the continuous flow separation device.

The apparatus can also have a controller for controlling fluid flow through the apparatus, and controlling the operation of the draw pump. The controller can also monitor (e.g., based on the number of revolutions of the draw pump) a total volume of whole blood drawn from the source, and may stop the draw pump when the target whole blood volume (e.g., 400 mL, 450 mL, or 500 mL) is withdrawn. The continuous flow separation device may include a centrifugal bowl that has the inlet, first blood component outlet, and second blood component outlet.

In some embodiments, the first blood component pump may reintroduce the first blood component within the first blood component storage device into the continuous flow separation device. The separation device can include an optical sensor that monitors the fluid within the continuous flow separation device as the first blood component is reintroduced into the continuous flow separation device. The controller may control the operation of the first blood component pump based upon an output of the optical sensor.

The first blood component storage container may collect pre-washed first blood component, and the first blood component pump may reintroduce pre-washed first blood component within the first blood component into the continuous flow separation device. The apparatus may also include an additive solution container containing additive solution, and an additive solution line fluidly connecting the additive solution container and first blood component outlet of the continuous flow separation device. The first blood component pump may draw additive solution from the additive solution container through the additive solution line and into the continuous flow separation device to wash the first blood component within the continuous flow separation device. The first blood component pump may draw washed first blood component from the continuous flow separation device and into the final first blood component collection container. The apparatus may also include filter to filter the washed first blood component.

The apparatus can also include a second blood component pump for recirculating second blood component within the second blood component storage container to the continuous flow separation device, and a second blood component recirculation line fluidly connecting the second blood component container and the first blood component outlet. A line sensor can monitor fluid flowing out of the continuous flow separation device as the second blood component is reintroduced/recirculated, and output a signal representative of the fluid. The controller may receive the output signal and control the operation of the second blood component pump based, at least in part, upon the output signal. The source may be a donor, and the apparatus may include an anticoagulant storage container, and an anticoagulant line fluidly connected to the draw line for introducing anticoagulant into the drawn whole blood.

In still further embodiments of the present invention, a method for separating whole blood can include drawing whole blood from a source, and introducing, using a draw pump, the whole blood into a continuous flow separation device that has an inlet, a first blood component outlet, and a second blood component outlet. The whole blood may be introduced into the continuous flow separation device through the inlet, and may be separated in the continuous flow separation chamber into a first blood component, a second blood component, and a third blood component while blood is being drawn from the source. The method may also include extracting the first blood component from the continuous flow separation chamber through the first blood component outlet, collecting the extracted first blood component within a first blood component storage container, and collecting the second blood component within a second blood component storage container (e.g., while extracting the first blood component).

The method may monitor a total volume of whole blood drawn from the source to determine when a target whole blood volume is drawn, and stop the draw of whole blood from the source when the target whole blood volume is reached. Once the draw is stopped, the method may recirculate at least a portion of the collected second blood component into the continuous flow separation device to displace the third blood component from the bowl, and collect the third blood component in a third blood component storage container.

Prior to recirculating the second blood component, the method may reintroduce at least a portion of the first blood component collected within the first blood component storage container into the continuous flow separation device to force additional second blood component out of the continuous flow separation device via the second blood component outlet. The method may then collect the additional second blood component within the second blood component storage container. Additionally, the method may monitor, using a bowl sensor, the amount of first blood component within the continuous flow separation device, and stop the reintroduction of first blood component from the first blood component storage container when the bowl sensor detects that the first blood component has reached a particular radius and that the continuous flow separation device is filled with appropriate amount of first blood component.

After collecting the third blood component, the method may reintroduce the first blood component collected within the first blood component storage container into the continuous flow separation device to force additional second blood component out of the continuous flow separation device via the second blood component outlet. The method may then collect the additional second blood component within the second blood component storage container. A line sensor can monitor the fluid flowing out of the continuous flow separation device, and output a signal representative of the fluid. The method may then stop the reintroduction of first blood component from the first blood component storage container when the line sensor detects cellular material.

The method may also include drawing additive solution from an additive solution container and into the continuous flow separation device. The additive solution may wash the reintroduced first blood component within the continuous flow separation device, and the method can extract the washed first blood component from the continuous flow separation device. The washed first blood component may be collected within a final first blood component container. The first blood component and the washed first blood component may be extracted using a first blood component pump.

In some embodiments, the method may then determine if additional first blood component remains within the first blood component container, and transfer the additional first blood component within the first blood component container to the continuous flow separation chamber. The method may then draw additional additive solution from the additive solution container to wash the additional first blood component, extract the washed additional first blood component from the continuous flow separation device, and collect the washed additional first blood component within the final first blood component container.

### BRIEF DESCRIPTION OF THE DRAWINGS

The foregoing features of embodiments will be more readily understood by reference to the following detailed description, taken with reference to the accompanying drawings, in which:

FIG. 1 is a schematic diagram of an automated whole blood separation system, in accordance with one embodiment of the present invention.

FIG. 2 is a schematic diagram of a disposable set for use with the system of FIG. 1, in accordance with one embodiment of the present invention.

FIG. 3 schematically shows a side view of a centrifuge bowl for use with the whole blood separation system of FIG. 1, in accordance with some embodiments of the present invention.

FIG. 4 is a flow chart depicting a method for separating whole blood, in accordance with one embodiment of the present invention.

FIG. 5 is a flow chart depicting an alternative method for separating whole blood, in accordance with additional embodiments of the present invention.

FIG. 6A schematically shows a cross-section view of a continuous flow centrifuge bowl for use with continuous flow whole blood separation systems, in accordance with some embodiments of the present invention.

FIG. 6A schematically shows a cross-section view of an alternative continuous flow centrifuge bowl for use with continuous flow whole blood separation systems, in accordance with some embodiments of the present invention.

FIG. 7 is a schematic diagram of a continuous flow whole blood separation system, in accordance with various embodiments of the present invention.

FIG. 8 is a flow chart depicting a method for separating whole blood using the system shown in FIG. 7, in accordance with various embodiments of the present invention.

FIG. 9 is a schematic diagram of an alternative continuous flow whole blood separation system, in accordance with various embodiments of the present invention.

FIG. 10 is a schematic diagram of a further embodiment of a continuous flow whole blood separation system, in accordance with various embodiments of the present invention.

FIG. 11 is a flow chart depicting a method for separating whole blood using the system shown in FIG. 10, in accordance with various embodiments of the present invention.

FIG. 12 is a schematic diagram of a three component continuous flow whole blood separation system, in accordance with various embodiments of the present invention.

FIG. 13 is a flow chart depicting a method for separating whole blood using the system shown in FIG. 12, in accordance with various embodiments of the present invention.

FIG. 14 is a schematic diagram of an alternative three component continuous flow whole blood separation system, in accordance with various embodiments of the present invention.

### DETAILED DESCRIPTION OF SPECIFIC EMBODIMENTS

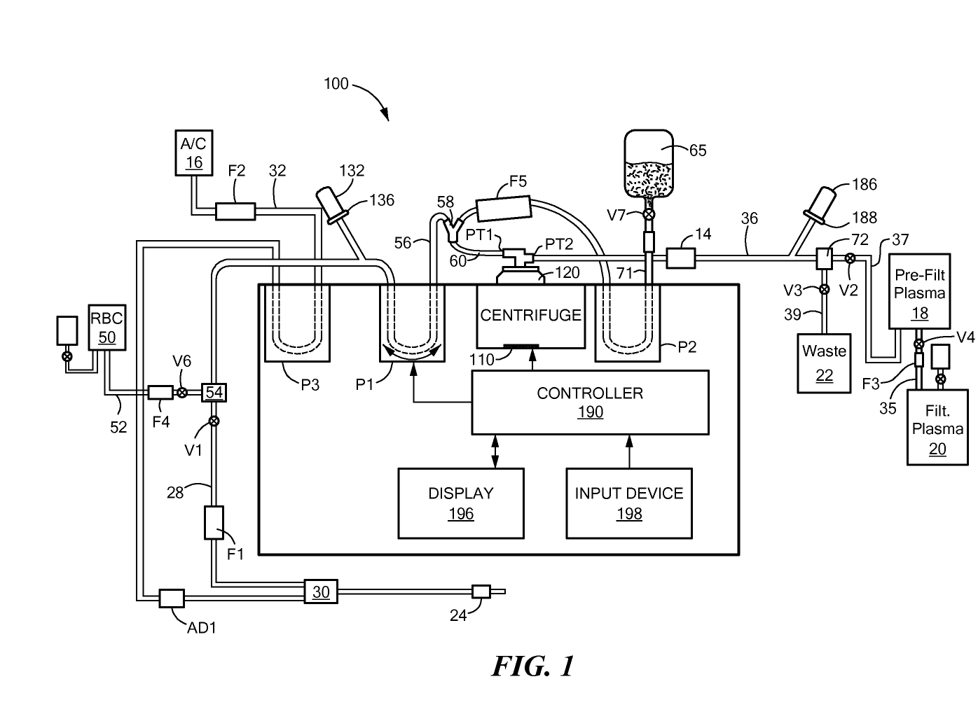

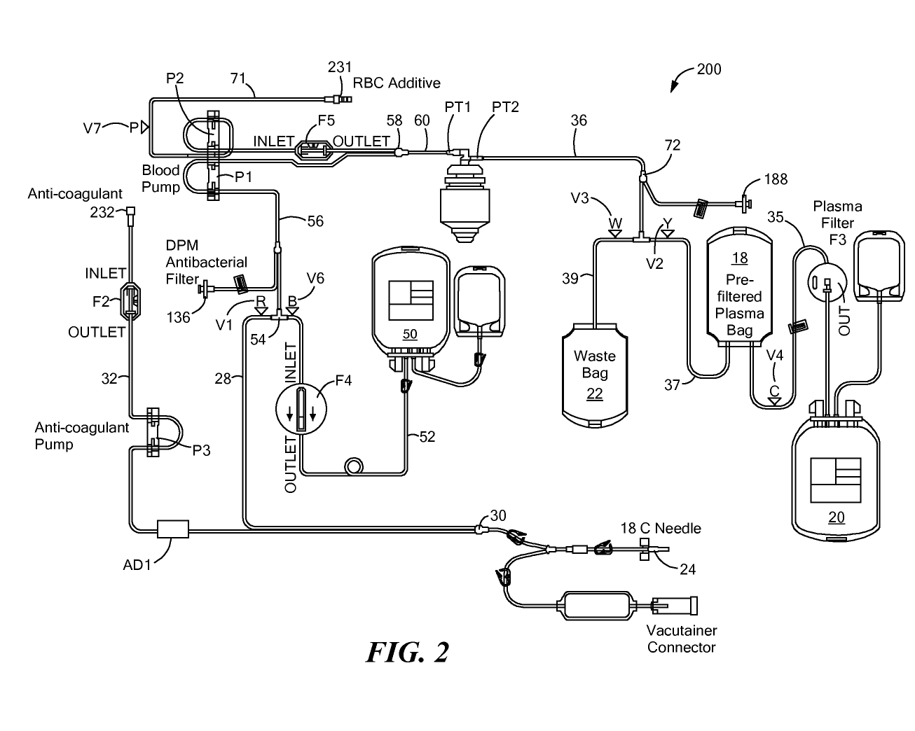

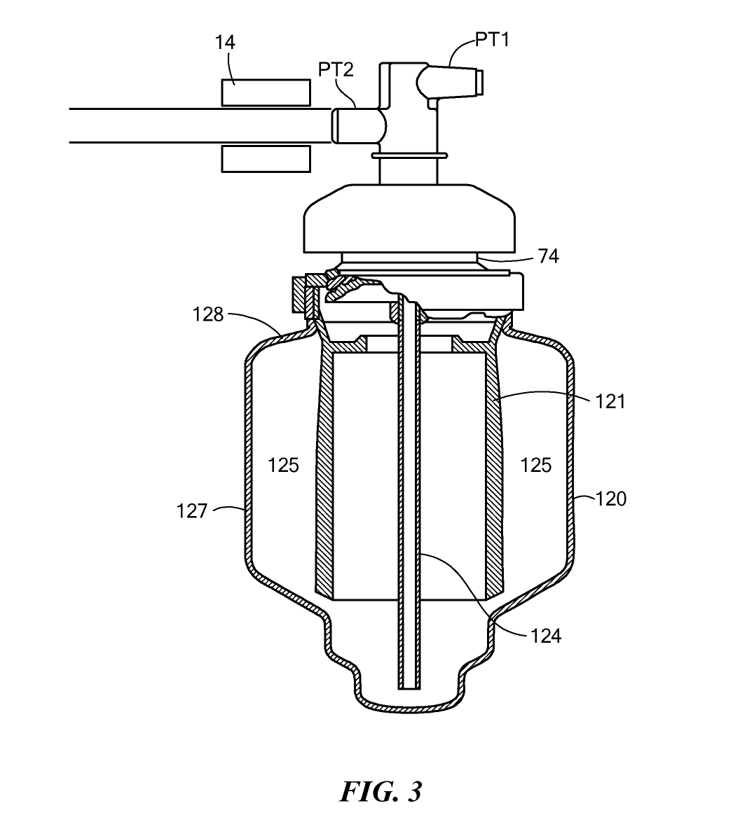

Referring to FIGS. 1 and 2, an automated whole blood collection system 100 uses a centrifuge 110, such as the centrifuge bowl described within U.S. Pat. No. 4,983,158, which is hereby incorporated by reference, to separate whole blood into its constituent components. Other types of separation chambers and devices may be used, such as, without limitation, a standard Latham type centrifuge, as described in U.S. Pat. Nos. 3,145,713 and 5,882,289, which are hereby incorporated by reference. The centrifuge 110 includes a rotating bowl 120 and stationary input and output ports PT1 and PT2 that are typically closely coupled to the bowl interior by a rotary seal 74 (see FIG. 3). Although the material and size of the bowl 120 may vary depending upon the application and amount of whole blood to be processed and/or red blood cells to be collected, preferred embodiments of the present invention utilize bowls having volumes ranging from preferably 200 to 300 ml, more preferably from 210 to 275 ml, more preferably from 220 to 230 ml, and most preferably about 225 ml. It should be noted that other bowl sizes may be utilized. For example, the bowl may be smaller than 210 ml, or larger than 300 ml. A preferable bowl material is K resin.

As shown in FIG. 3, in some embodiments, the centrifuge bowl 120 may include a core 121 located within the interior of the bowl 120, such as the centrifuge bowl described within U.S. Pat. No. 4,943,273, which is hereby incorporated by reference. As fluid to be processed (e.g., whole blood, etc.) enters the bowl 120 through input port PT1, the fluid flows through feed tube 124 and into the bottom of the bowl 120. The centrifugal forces then force the fluid to flow outwardly and upwardly into a separation region 125. As discussed in greater detail below, if the collected blood components (e.g., red blood cells) are to be washed, as the wash solution (or additive solution) enters the bowl 120, the wash solution may similarly flow through the feed tube 124, into the bottom of the bowl 120, and into the separation region 125.

The input port PT1 of the centrifuge bowl 120 is in fluid communication with a venous access device 24 (e.g., a phlebotomy needle) via a tubes 28, 56 and 60, and Y-connectors 30, 54 and 58 when a valve V1 is open. As discussed in greater detail below, the venous access device 24 may be replaced with a whole blood bag (not shown) in case the whole blood is to be first pooled and then supplied (or otherwise collected/stored prior to processing). The tube 28 has compatibility with blood, as is all the tubing in the system 100. The outlet port PT2 of the centrifuge 110/bowl 120 is selectively coupled by a tube 36, a valve V2, connector 72, and a tube 37 with a first container 18 labeled pre-filtered plasma. A second container 22 labeled waste is selectively coupled to the outlet port PT2 via the tube 36, a valve V3 and a tube 39. Additionally, a third container 20 (e.g., for filtered plasma) may be selectively coupled to the pre-filtered plasma container 18 via a tube 35, a valve V4 and a filter F3. Each of the containers 18/20/22 may be suspended by weight scales (not shown).

A bag or container 16 for storing an anticoagulant is in fluid communication with the venous access device/phlebotomy needle 24 via a bacteria filter F2, a tube 32, and the Y-connector 30. The tube 32 may also include an air detector AD1 that detects the presence of air bubbles within the anticoagulant line 32. The bacteria filter F2 prevents any bacteria in the anticoagulant container 16 (or introduced into the container 16/anticoagulant when the container 16 is connected to connection 232) from entering the system. Containers 16, 18, 20, and 22 are preferably plastic bags made of a blood compatible material.

In order to monitor the pressure within the system 100, the system may also include one or more pressure sensors. For example, the system 100 may include a pressure sensor on tube 56 to measure the pressure between the pump P1 and the source of whole blood (e.g., the subject or whole blood container). Similarly, the system 100 may also include a pressure sensor connectable to tube 36 to measure the pressure between the centrifuge bowl 120 and the waste container 22/pre-filtered plasma container 18. Positioned between each of the pressure sensors and tubing 32 may be a filter 136/188 (e.g., a 0.2 μm hydrophobic filter and/or anti-bacterial filter) to preserve sterility of the system 100 from the blood and biological material that may be in tubing 32

The filters 136/188 may be located at the end of a tubing line and may include a housing (e.g., a plastic housing) containing a filter membrane. During set-up of the system 100, one end of the filter 132/186 may be inserted into a port located on the system 100 to connect the filter 136/188 with the associated sensor which, in turn, may be contained within the system 100.

The system 100 may also have a red blood cell collection container 50 (RBC container) for storing the red blood cells collected during whole blood processing. The RBC container 50 may be fluidly connected to the bowl 120 via the line 52, filter F4, valve V6, connector 54, line 56, connector 58 and line 60. Like the other containers, the RBC container 50 is also preferably a plastic bag made of blood compatible material. As discussed in greater detail below, after the whole blood processing is complete, red blood cells within the bowl 120 are transferred to the RBC container 50 for storage and/or further processing.

In some embodiments, the system 100 may also include a controller 190 that controls the overall operation of the system 100 and centrifuge 110. For example, the controller 190 may control the operation of peristaltic pumps P1, P2, and P3 as well as, valves V1/V2/V3/V4/V6/V7 to control the direction and duration of flow through the system 100. The controller 190 may also be coupled to a display screen 196 that presents information to a system operator, and an input device 198 that allows the system operator to input information and supply the controller 190 with information. For example, the input device 198 may allow the user to input a target volume of whole blood to withdraw, a target volume of red blood cells to collect, and/or the hematocrit value of the whole blood being drawn into the system 100. As discussed in greater detail below, the controller 190 may control the speed of the centrifuge 110 based, at least in part, upon the hematocrit valve of the whole blood (e.g., the controller may increase the speed for high hematocrit donors). The controller 190 may also receive outputs from the pressure sensors and a line sensor 14. The line sensor 14 may be an optical sensor that detects the presence of blood components passing through the line sensor 14 from the output port PT2.

As shown in FIG. 2, various components may be packaged together as a disposable set 200. For example, the disposable set 200 may include tubes 28/52/56/32/60/36/37/35/39/71, connectors 30/54/5/72, valves V1/V2/V3/V4/V6/V7, the centrifuge bowl 120, filters F1/F2/F3/F4/F5, the waste container 22, the pre-filtered plasma bag 18, the filtered plasma bag 20, and the red blood cell (RBC) storage bag 50. Additionally, the disposable set 200 may also include connection ports for the anticoagulant container 16 and the additive container 65. For example, the disposable set 200 may include a first sterile connection 231 for connecting the red blood cell (RBC) additive solution container 65, and a second sterile connection 232 for connecting the anticoagulant container 16. Prior to starting the whole blood separation procedure, the disposable set 200 may be removed from its packaging and installed into the system 100, as shown in FIG. 1.

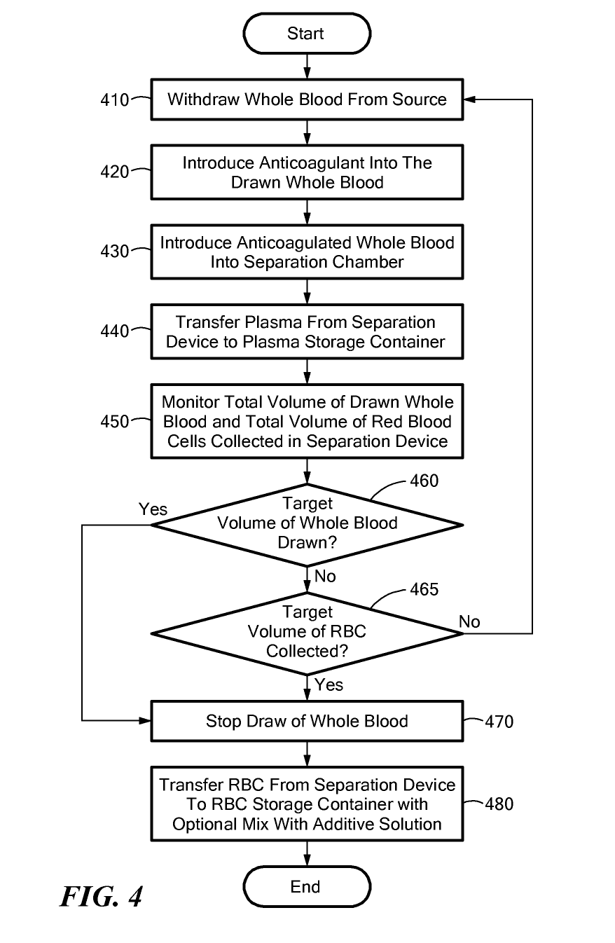

FIG. 4 is a flowchart depicting a method for separating whole blood in accordance with one embodiment of the present invention. Once the disposable set is installed into the system 100 and the anticoagulant and additive containers 16/65 are connected to their respective sterile connections 232/231, the system may begin to withdraw whole blood from the subject (Step 410). As the whole blood flows through tube 28, the anticoagulant pump P3 mixes the anticoagulant from the container 16 with the whole blood (Step 420) (e.g., anticoagulant may flow through line 32 and mix with the whole blood at connector 30). Additionally, valve V1 is open, allowing the anticoagulated whole blood to pass through the tube 28 and blood filter F1 (optional) before being pumped into the centrifuge bowl 120 through the inlet port PT1. It should be noted that the blood filter F1 is optional, and some embodiments (e.g., single cycle embodiments) may not utilize a blood filter F1.

As discussed above, the whole blood is introduced into the bottom of the bowl 120 though the feed tube 124 (Step 430). Additionally, it should be noted that the ratio of the anticoagulant to whole blood is typically about 1:10, but some embodiments may use other ratios, for example, 1:8. The operation of each of the pumps and valves in the system 100 can be performed in accordance with desired protocols under the control of the controller 190, which may be, for example, a microprocessor.

As the bowl 120 is rotated, centrifugal forces will force the anticoagulated whole blood into the separation region 125 and separate the whole blood into a number of blood components (e.g., red blood cells and plasma). The number of rotations of the bowl 12 can be selected, for example, within a range of 5500 to 7,500 rpm. The blood is separated into different fractions in accordance with the component densities. The higher density component, i.e., red blood cells, is forced to the outer wall 127 of the bowl 120 while the lower density plasma lies nearer the core 121. A buffy coat may form between the plasma and the RBC. The buffy coat is made up of an inner layer of platelets, a transitional layer of platelets and white blood cells, and an outer layer of white blood cells. The plasma is the component closest to the outlet port from the separation region and is the first fluid component displaced from the bowl 120 via the outlet port PT2 as additional anticoagulated whole blood enters the bowl 120 through the inlet port PT1.

Returning to FIG. 1, as additional whole blood enters the bowl 120, the displaced plasma passes through the line sensor 14, the tube 36, the valve V2 (in the open position), connector 72, and line 37, and enters the pre-filtered plasma container 18 (Step 440). The plasma entering the pre-filtered plasma container 18 may later be filtered through filter F3 and stored in filtered plasma container 20.

As the anticoagulated whole blood is introduced into the bowl 120 and the plasma is displaced (e.g., transferred) to the pre-filtered plasma container 18, the controller may monitor/calculate (1) the total volume of whole blood drawn from the source, and (2) the volume of red blood cells collected within the bowl 120 (Step 450). For example, the controller 190 may calculate the total volume of blood drawn from the source based upon the number of revolutions of the draw pump P1 (e.g., the total volume of donor whole blood drawn equals the number of revolutions multiplied by the known amount of volume per revolution and adjusted for anticoagulation ratio).

Additionally, the controller 190 may also monitor the output signal from the optical sensor 14 to determine when the bowl 120 is full of red blood cells. For example, the optical sensor 14 may monitor the fluid exiting the bowl 120 through the outlet PT2 and detect when the fluid changes from one component to the next. The optical sensor 14 can detect when the fluid exiting the bowl 120 changes from plasma to the buffy coat, and from the buffy coat the red blood cells. Once sensor 14 determines that the fluid exiting the bowl 120 is buffy coat cells, the controller 190 will consider the bowl 120 to be full and the target volume of red blood cells collected.

Additionally or alternatively, the bowl 120 may have a sensor (e.g., an optical sensor) located on a shoulder portion 128 of the bowl 120. The shoulder mounted sensor may monitor each layer of the blood components as they gradually and coaxially advance toward the core 121 from the outer wall 127 of the bowl 120. The shoulder sensor may be mounted in a position at which it can detect the red blood cells (or the buffy coat) reaching a particular radius. The controller 190 may then monitor the output from the shoulder sensor to determine when the bowl 120 has appropriate amount of red blood cells.

In some embodiments, the controller 190 may alternatively determine when the bowl 120 is full of red blood cells based upon the amount of whole blood drawn and the hematocrit of the whole blood. For example, the amount of red blood cells collected within the bowl 120 should be substantially equal to the volume of whole blood drawn from the source multiplied by the hematocrit of the drawn blood.

As mentioned above, the total amount of whole blood that may be safely withdrawn from a subject/patient is limited (e.g., typically 400 ml+/−10%, 450 ml+/−10%, or 500 ml+/−10%). As also discussed above, the volume of whole blood processed and the hematocrit value of the whole blood determines (along with the system efficiency) the volume of red blood cells that may be collected. For example, processing 400 ml whole blood with a hematocrit of 35% will yield less red blood cells than processing the same volume of whole blood with a hematocrit of 60%.

To that end, various embodiments of the system 100, take into account the maximum volume of whole blood that may be withdrawn (e.g., a target volume) as well as the target volume of red blood cells desired when processing whole blood (e.g., the maximum volume of the bowl 120). For example, in some embodiments of the present invention, as the controller 190 monitors the volume of whole blood drawn and the volume of red blood cells collected in the bowl 120 (e.g., based upon the output from the line sensor 14 or shoulder sensor), the controller 190 may compare the values to the target values. In other words, the controller 190 may compare the current volume of drawn whole blood to the target/maximum value (Step 460). Similarly, the controller 190 may monitor the output from the optical sensor 14 (or shoulder sensor) to determine when the bowl 120 is full of red blood cells (indicating that the target volume of red blood cells has been collected) (Step 465). If either the target volume of whole blood has been drawn from the source or the target volume of red blood cells has been collected (e.g., the bowl 120 is full of red blood cells), the controller 190 may stop drawing whole blood from the donor/source (Step 470) and stop the bowl 120 from spinning. Once the controller 190 has stopped drawing whole blood and the bowl 120 has stopped spinning, the controller 190 may then transfer some or all the red blood cells contained within the bowl 120 to the RBC storage container 50 (e.g., by reversing draw pump P1 and drawing the red blood cells though tube 60, connector 58, tube 56, connector 54, valve V6, filter F4, tube 52 and into the RBC container 50) (Step 480). Accordingly, because the bowl 120 is stopped to collect the red blood cells, this system 100 is a discontinuous system.

As the controller 190 transfers the red blood cells within the bowl 120 to the RBC storage container 50, the system 100 may also introduce additive solution from the RBC additive solution container 65 into the red blood cells. For example, as discussed above, the controller 190 may energize pump P2 and draw additive solution from container 65, through valve V7, tube 71, and filter F5. The additive solution may then mix with the red blood cells being transferred at connector 58. Additionally or alternatively, the additive solution may be introduced into the bowl and mixed with the red blood cells within the bowl 120 prior to the transfer of the red blood cells to the RBC storage container 50.

It should be understood that, by monitoring the amount of whole blood drawn from the subject, monitoring the amount of red blood cells collected within the bowl 120, and stopping the draw step when a target value is reached for either, various embodiments of the present invention are able to maximize the amount of red blood cells collected yet ensure that the maximum allowable volume of whole blood is not exceeded. Additionally, unlike prior art systems, various embodiments of the present invention may be performed “chair-side” and are able to utilize a single fixed-volume bowl 120 which reduces the cost and complexity of the system. For example, unlike variable volume separation chambers, embodiments of the present invention that use fixed volume chambers do not require a separate control system to regulate chamber volume, are less expensive to manufacture, and have reduced procedure times.

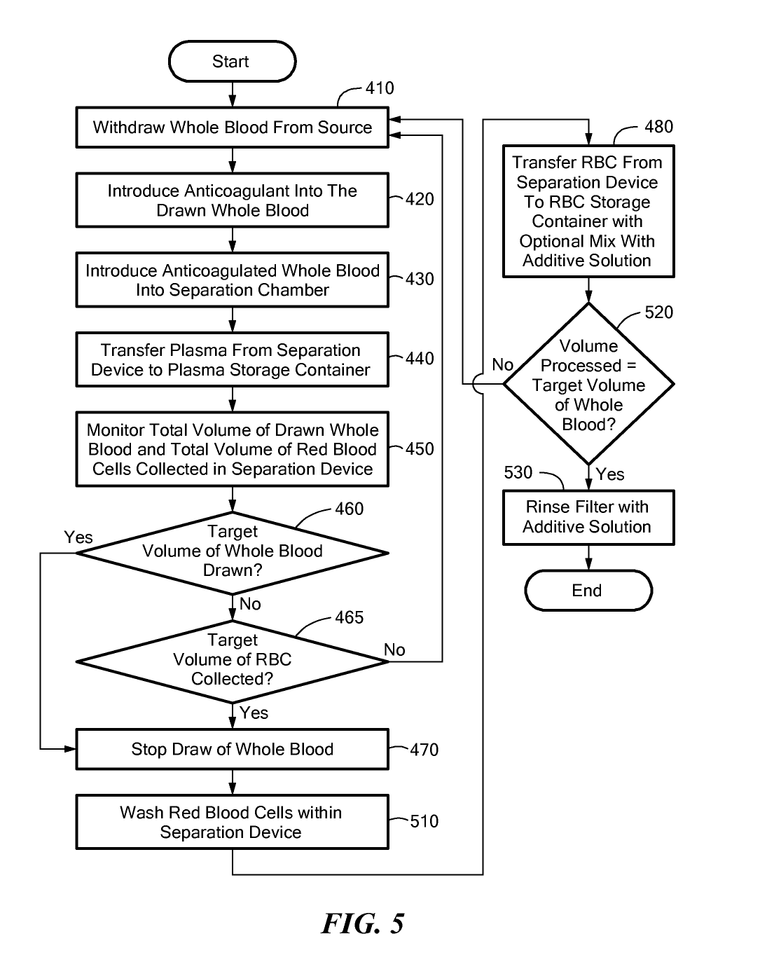

In addition to the steps shown in FIG. 4, some embodiments of the present invention may have additional optional steps. For example, as shown in FIG. 5, prior to transferring the collected red blood cells to the RBC container 50, the system 100 may wash the red blood cells to remove proteins within the red blood cells (Step 510). To that end, the system 100/controller 190 may energize pump P2 to draw additive solution from container 65 through valve V7, line 71, filter F5, connector 58, and line 60 and into the bowl 120 (e.g., through inlet port PT1). As additional additive solution enters the bowl 120 (e.g., when the bowl 120 is spinning), the wash solution/protein mixture will be displaced from the bowl 120 through the outlet port PT2 and will be sent to the waste container 22 (e.g., through line 36, connector 72, valve V3, and line 39). Once the wash step is completed, the system 100 may then transfer the washed red blood cells within the separation device to the RBC storage container 50 (Step 480). During the transfer, the system 100 may also (optionally) add the additive solution required for storage of the red blood cells. It should be noted that, as the system 100 transfers the red blood cell/additive solution mixture, the mixture may pass through a leukoreduction filter F4 which, in turn, removes white blood cells (e.g., leukocytes) from the red blood cells.

Additionally, particularly for those embodiments in which the whole blood was first collected in a whole blood bag (e.g., those embodiments in which the source is a whole blood bag), after transferring the red blood cells to the RBC container 50, some embodiments may process additional whole blood in order to collect additional red blood cells if the total volume of whole blood processed is less than the target volume (or less than that collected within the whole blood bag) (Step 520). For example, the system 100/controller 190 may determine if there is sufficient whole blood remaining in the whole blood bag to start a second red blood cell collection cycle. If the system 100 determines there is a sufficient amount of whole blood, the system 100 may, once again, draw whole blood from the whole blood bag and into the bowl 120 and continue to process the whole blood in a manner similar to that described above until either the target volume of whole blood is reached or the target volume of red blood cells is collected within the bowl (for a second time) (e.g., the system may repeat steps 410, 420, 430, 440, 450, 460, 465, and 470).

In addition to or instead of determining if there is a sufficient volume of whole blood within the whole blood bag prior to drawing additional whole blood from the whole blood bag, the system 100 may determine whether there is a sufficient volume of red blood cells remaining within the whole blood bag. For example, the controller 190 can calculate the volume of red blood cells remaining within the whole blood bag based upon the volume of whole blood remaining (e.g., the initial volume of whole blood in the whole blood bag minus the volume of whole blood already drawn/processed) and the hematocrit of the whole blood (e.g., the volume of red blood cells remaining will be equal to the volume of whole blood remaining in the whole blood bag multiplied by the hematocrit of the whole blood). If the system 100/controller 190 determines there is a sufficient volume of red blood cells remaining within the whole blood bag, the system 100/controller 190 may start the second collection cycle.

Additionally, in order ensure that there is sufficient free volume in the bowl 120 to process the second cycle of whole blood, the system 100 may transfer some or all of the red blood cells within the bowl 120 to the red blood cell storage bag 50. For example, the system 100 may transfer all of the red blood cells to the storage bag 50 or the system 100 may transfer just enough red blood cells to accommodate the additional red blood cells that will be collected within the bowl 120 during the second cycle (e.g., the volume of red blood cells remaining in the whole blood bag).

Once the red blood cell/additive solution mixture is transferred to the RBC container 50, the system 100 may perform a rinse step to flush any red blood cells trapped in the filter F4 through the filter F4 and into the RBC container 50 (Step 530). For example, the controller 190 may energize pump P2, and transfer additional additive solution (e.g., 70 mL) from container 65 through line 71, line 56, line 52, and through filter F4. As the additive solution passes through the filter F4, it flushes the trapped red blood cells out of the filter F4 and into the RBC container 50 which, in turn, increases the RBC recovery.

In some instances, various embodiments of the present invention may be able to increase the volume of plasma that is collected and/or increase the hematocrit of the final red blood cell product by adjusting/increasing the speed to the centrifuge bowl 120. For example, when processing whole blood with a relatively high hematocrit, the controller 190 may increase the speed of the centrifuge 110. By increasing the speed of the centrifuge 110, additional plasma may be separated from the incoming whole blood (e.g., the efficiency of the separation will be increased) and collected. Additionally, in some high hematocrit donors, the hematocrit of the final red blood cell product will increase (e.g., because the red blood cells within the bowl 120 will pack more tightly against the wall 127 of the bowl).

As discussed above, for a discontinuous process, various bowl sizes may be used within the embodiments of the present invention. To that end, in some embodiments, it is desirable to choose a bowl 120 having a volume that is able to accommodate an appropriate volume of red blood cells (e.g., based on the hematocrit of the whole blood to be processed and the source of the whole blood). For example, for those embodiments in which the source is a whole blood bag, it is preferable to choose a bowl size that may accommodate the red blood cell volume of the lowest hematocrit donor. For those embodiments in which the source is a donor, it may be preferable to choose a bowl size that may hold the target volume of red blood cells (e.g., 180 ml). It is preferable that a bowl size is chosen that can accommodate from 150-210 ml of red blood cells, more preferably 160 to 200 ml of red blood cells, more preferably 170 to 190 ml of red blood cells, and most preferably around 180 ml of red blood cells.

However, in alternative embodiments, a smaller bowl 120 may be used. In such embodiments, the processing may occur in multiple steps. For example, the system 100 may draw a volume of whole blood (e.g., less than the target volume of whole blood), process the blood, and collect the red blood cells and plasma. The system 100 may then repeat the draw/processing steps until the target volume of whole blood is drawn or the target volume of red blood cells is collected.

Although a discontinuous system is described above, other embodiments of the present invention can utilize a continuous flow centrifuge bowl that allows multiple blood components (e.g., red blood cells and plasma) to be removed/collected simultaneously. In this manner, various continuous flow embodiments described herein can avoid the batch-like/intermittent processing of the discontinuous systems. To that end, various continuous flow embodiments utilize a centrifuge bowl having an additional outlet to allow for the extraction of the first and second blood components (e.g., red blood cells and plasma) as whole blood is introduced into the bowl.

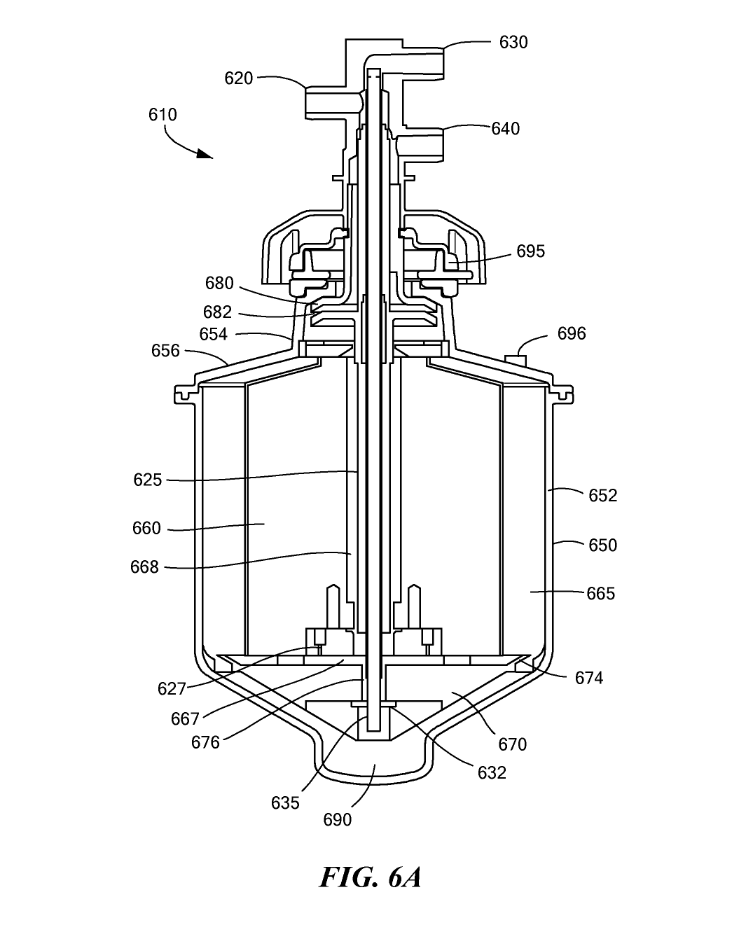

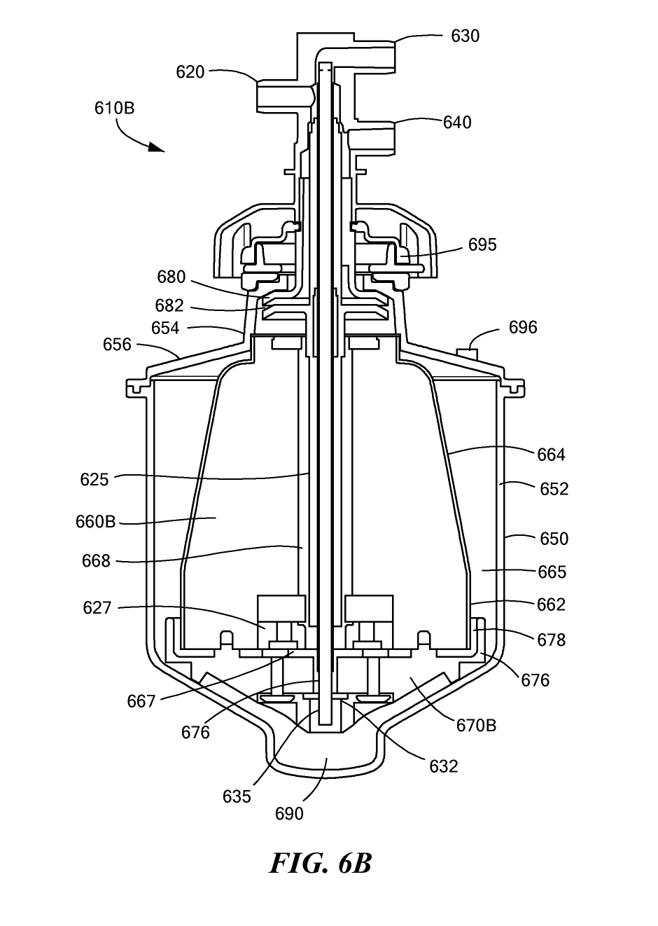

For example, FIG. 6A shows a cross-sectional view of an exemplary continuous flow centrifuge bowl 610 that may be used in various continuous flow embodiments of the present invention. As shown in FIG. 6A, unlike the bowl shown in FIG. 3, the continuous flow bowl 610 can have an inlet port 620, a first blood component outlet 630, and a second blood component outlet 640. Additionally, the bowl 610 may have an outer body 650 that defines the structure of the bowl 610 and an inner volume into which the whole blood may be introduced for processing. The outer body 650, in turn, includes a main wall 652, a neck portion 654, and shoulder portion 656 that connects the main wall 652 and the neck portion 654.

Within the interior of the outer body 650, the bowl 610 can include a number of cores that displace some of the volume within the outer body 650, and create separation region(s) in which the whole blood separates. For example, the bowl 610 may include an upper core 660 that fills a significant portion of the inner volume. The upper core 660 and the main wall 652 of the body 650 may define a separation region 665 in which the whole blood introduced into the bowl 610 will separate into its individual components (e.g., red blood cells, plasma, etc.). Additionally, the upper core 660 can have a shaft 668 extending through the center. As discussed in greater detail the shaft 668 may serve as a channel through which a number of tubes (e.g., an inlet tube and an extraction tube) can pass.

The bowl 610 may also include a lower core 670 located below the upper core 660 (e.g., distal to the upper core 660). The lower core 670 may have a flange 674 that extends outward and slightly upward (e.g., proximally) from the lower core 670. The flange 674 may extend from the outer diameter of the lower core 670 and may extend such that it is radially outward from the upper core 660. During operation, the flange 674 may help prevent whole blood from flowing into the region below the lower core 670 (e.g., where the red blood cells are extracted from).

Within the neck portion 654 of the outer body 650, the centrifuge bowl 610 can have an effluent skirt 680 extending radially outward from the center of the bowl 610. The effluent skirt 680 may have an effluent channel 682 that extends through the skirt 680, and that is fluidly connected to the second blood component outlet 640. To that end, various embodiments are able to extract and collect blood components (e.g., plasma and platelets) through the effluent skirt 680 and the second blood component outlet 640 (e.g., the blood component exiting the bowl can flow through the effluent channel 682 and the second blood component outlet 630 to exit the bowl 610).

As mentioned above, in order to facilitate the transfer of fluids (e.g., whole blood and blood components) in and out of the centrifuge bowl 610, the bowl 610 can have an inlet and multiple outlets (e.g., a first blood component outlet 630 and a second blood component outlet 640). As the name suggests, the inlet 620 may be used to introduce whole blood into the bowl 610. In many blood processing procedures, it is desirable to introduce the whole blood into an area near the bottom of the bowl 610. To that end, the bowl 610 may include an inlet tube 625 that extends downward from the inlet 620, through the shaft 668, and into an introduction region 667 (where the whole blood is introduced into the bowl 610) located between the upper core 660 and the lower core 670. In order to prevent whole blood (or other fluid) from flowing into the shaft 668 (and bypassing the separation region), some embodiments may include a bypass seal 627 that isolates the introduction region 667 from the shaft 668 in the upper core 660. To allow rotation of the bowl 610, the bypass seal 210 can be a rotary seal.

In addition to the inlet 620, the bowl 610 can also include a first blood component outlet 630 and a second blood component outlet 640. The first blood component outlet 630 can be used to remove a first blood component (e.g., red blood cells) from the bowl 610. Additionally, in a manner similar to the inlet 620, the first blood component outlet 630 may be fluidly connected to a tube (e.g., an extraction tube 635) that extends downward from the first blood component outlet 630, through the shaft 668, through an opening 676 in the lower core 670, and into a first blood component extraction region 690 located below the lower core 670 (e.g., between the lower core 670 and the bottom of the bowl 610). As discussed in greater detail below, red blood cells collect below the lower core 670 and may be extracted from the bowl 610 via the extraction region 690.

To prevent leakage past the lower core 670 (e.g., through opening 676), the lower core 670 can also have a seal 632 (e.g., a rotary seal) between the first blood component extraction tube 635 and the opening 676. As discussed in greater detail below, a pump can draw the first blood component out of the first blood component extraction region 690, through the first blood component extraction tube 635 and out of the first blood component outlet 630.

The second blood component outlet 640 may be used to remove the second blood component (and perhaps a third blood component) from the bowl 610. To that end, the second blood component outlet 640 may be fluidly connected to the separation region 665 (via the effluent channel 682). Therefore, when additional whole blood is added to the bowl 610, the second blood component is pushed towards the neck portion 654, and can flow out of the bowl 610 via the effluent channel 682 and the second blood component outlet 230.

Like the bowl shown in FIG. 3, the continuous flow centrifuge bowl 610 shown in FIG. 6A (and FIG. 6B) may include a rotary seal 695 that connects the ports (e.g., the inlet 620, first blood component outlet port 630, and second blood component outlet port 640) to the outer body 650 of the bowl 610. The rotary seal 695 allows the bowl 610 (and the upper core 660 and lower core 670) to spin while the inlet 620, first blood component outlet 630, and second blood component outlet 640 remain stationary.

During blood processing it may be important to know how full the bowl 610 is and how much of a given blood component is within the bowl 610. To that end, some embodiments may include an optical system 696 located on the shoulder 656 of the outer body 650. The optical system 696 may include an LED that emits a beam that illuminates a small area of the shoulder 656. Additionally, the optical system 696 may also include an optical sensor that is focused on the illuminated area of the bowl shoulder 656. As the various blood components (e.g., plasma, platelets, red blood cells) encroach on this illuminated area, the signal received back at the sensor changes based upon the characteristics (e.g., density) of the encroaching fluid. Based upon the change in sensor output, the system (e.g., the control system) will be able to determine how much of a given blood component resides within the bowl 610 and how full the bowl 610 is.

FIG. 6B shows an alternative embodiment of a continuous flow centrifuge bowl 610B. The centrifuge bowl 610B shown in FIG. 6B is similar to the centrifuge bowl 610 shown in FIG. 6A in many respects (e.g., it has an inlet 620, first blood component outlet 630, and second blood component outlet 640, etc.). However, the centrifuge bowl 610B shown in FIG. 6B can have different cores that allow the whole blood entering the bowl 610B to be separated into three components (e.g., red blood cells, platelets, and plasma). For example, unlike the upper core 660 in FIG. 6A (which has a straight side wall), the upper core 660B in FIG. 6B can have a side wall with a straight section 662 and an angled section 664 that slopes inward toward the center of the bowl 610B (e.g., such that the upper core 660B decreases in diamater from the top of the straight section 662 to the top surface of the upper core 660B).

Additionally, instead of the flange 674, the lower core 670B can include a vertical wall 676 that extends upward from the lower core 670B. As shown in FIG. 6B, the vertical wall 676 can be located radially outward from the straight section 662 of the upper core 660B and can create an annular space with the straight section 662. This annular space between the straight section 662 of the upper core 660B and the vertical wall 676 may act as a primary separation region 678 in which separation of the whole blood begins. Additionally, like the flange 674, the vertical wall 676 can prevent whole blood from entering the area below the lower core 670B. The lower cores 670 and 670B can be used interchangeably for two component or three component collection.

Like the discontinuous bowl 120 discussed above and shown in FIG. 3, the continuous flow bowls 610/610B can also have a range of volumes, depending on the target draw volume. For example, for a 400 mL target draw volume, some embodiments of the continuous flow bowls 610/610B can have a volume ranging from 150-300 mL, more preferably from 170 to 190 mL, and most preferable about 180 mL. However, the volume of the continuous flow bowl 610/610B may be different for a different target draw volume (e.g., for target draw volumes of 450 mL and 500 mL). A preferable bowl material for the continuous flow bowls 610/610B is Polycarbonate.

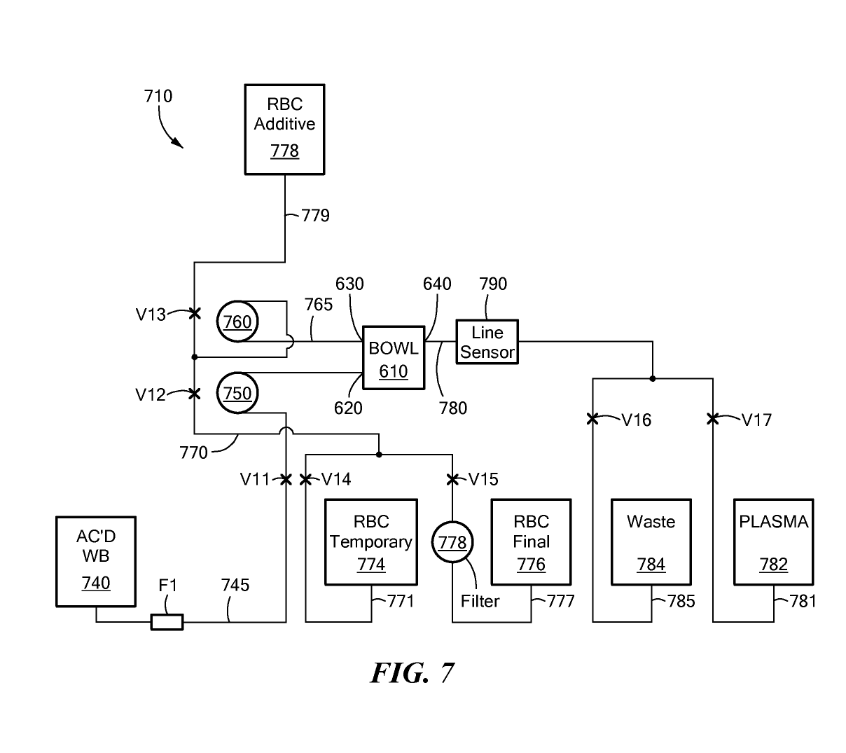

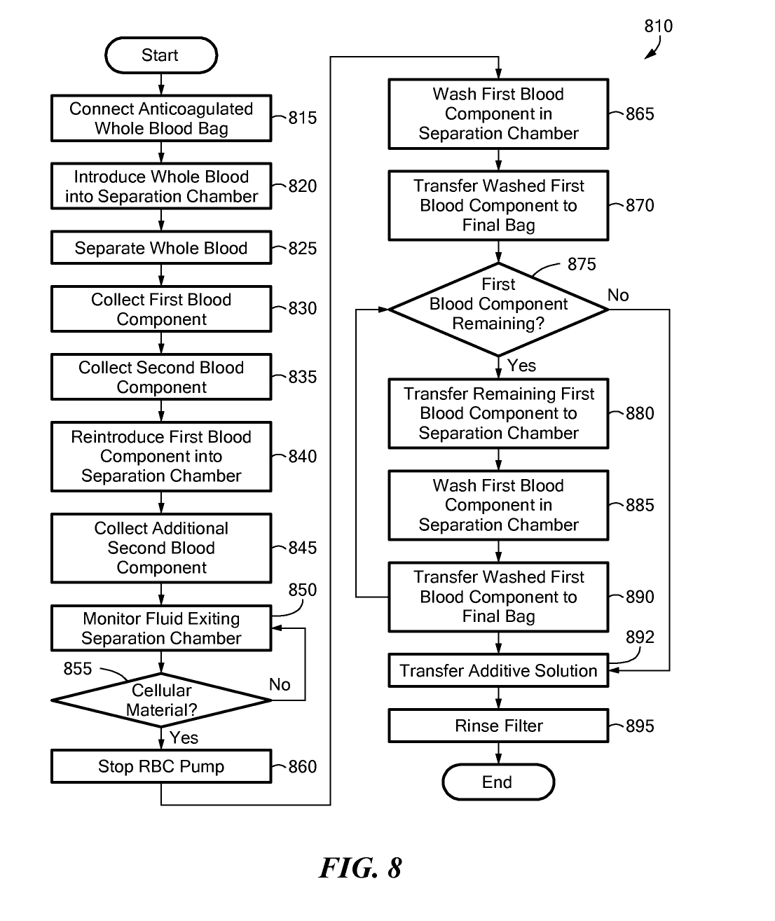

FIG. 7 schematically shows one embodiment of a continuous flow system 710 for separating whole blood and collecting the individual components. FIG. 7 will be discussed in connection with FIG. 8 which is a flow chart of an exemplary blood processing method using the system shown in FIG. 7. It is important to note that, this embodiment separates whole blood into two components (red blood cells and plasma), which are collected. To that end, this embodiment can utilize the centrifuge bowl 610 shown in FIG. 6A.

First, a user may connect a bag 740 containing anticoagulated whole blood to be processed (Step 815). Once connected, the system 710 can begin to draw the anticoagulated whole blood from the bag 740, through line 745, blood filter F1 (optional), and into the centrifuge bowl 610 (e.g., into the introduction region 667) using a draw pump 750 (Step 820). Once in the centrifuge bowl 610, the centrifugal force created by spinning the bowl 610 will cause the whole blood to enter the separation region 660 and separate into multiple components (e.g., red blood cells and plasma) (Step 825).

The system 710 may then begin collecting the red blood cells and plasma within the bowl 610. For example, the red blood cell pump 760 may begin drawing the red blood cells out of the bowl 610 (e.g., via the first blood component extraction tube 635 and the first blood component outlet 630), through lines 765, and 771 and, valves V12 and valve V14 and into a temporary red blood cell storage container 774 (Step 830). Additionally, at the same time, as additional whole blood is introduced into the bowl 610, plasma within the bowl 610 (e.g., in the separation region 660) is displaced out of the bowl 610 and flows through line 780 and valve V17, and into a plasma bag 782 where it is collected (Step 835). The plasma collected plasma container 782 may later be filtered and stored in filtered plasma container (not shown). The system 610 may continue the drawing, separation, and collection processes (e.g., Steps 820, 825, 830 and 835) until a target volume of whole blood has been processed (e.g., until there is no more whole blood within the whole blood bag 740).

Once the target volume of whole blood has been processed (e.g., the whole blood bag 740 is empty), the system 710 can then reintroduce a portion of the red blood cells collected within the temporary red blood cell container 774 back into the bowl 610 (Step 840). For example, the system 710 may operate the red blood cell pump 760 in the reverse direction to draw the first blood component out of the temporary red blood cell container 774, through line 771, and back into the bowl 610 via the first blood component outlet 630. As the red blood cells are reintroduced into the bowl 610, plasma remaining in the bowl 610 will be pushed out through the second blood component outlet port 640, through line 780 and valve V17 and into the plasma container 782, where the additional plasma is collected (Step 845).

It is important to note that as the plasma is exiting the bowl 610, a line sensor 790 located on line 780 will monitor the fluid exiting the bowl 610 (Step 850), and will detect when the fluid leaving the bowl 610 changes from plasma to a cellular material (e.g., platelets, red blood cells, etc.) (Step 855). When the fluid exiting the bowl 610 changes from plasma to cellular material, the system 710 will stop the red blood cell pump 760 to stop drawing the red blood cells from the temporary red blood cell bag 774 (Step 860). At this point, the bowl 610 will be filled primarily with red blood cells.

As shown in FIG. 7, the system 710 may also have a final red blood cell bag 776 and an additive solution bag 779 containing an additive solution. Both the final red blood component container 776 and the additive solution container 778 may be fluidly connected to the red blood cell pump 760. Once all of the plasma has been collected and the bowl 610 is full of red blood cells, the system 710 may wash the red blood cells (optional) with additive solution to remove proteins within the red blood cells (Step 865). To that end, the system 710 (e.g., the controller) may energize the red blood cell pump 760 to draw additive solution from container 778 through valve V13, line 779, and into the bowl 610 (e.g., through the first blood component outlet 630) to wash the red blood cells within the bowl 610 (Step 865). As additional additive solution enters the bowl 610 (e.g., when the bowl 610 is spinning), the additive solution/protein mixture will be displaced from the bowl 610 through the second blood component outlet port 640 and will be sent to a waste container 784 (e.g., through line 780, valve V6, and line 785).

Once the wash step is completed, the system 710 may then transfer the washed red blood cells within the bowl 610 to the final RBC storage container 776 (Step 870). For example, the system 710 may, once again, energize the red blood cell pump 760 to draw the washed red blood cells out of the bowl 610, though lines 770 and 777, and into the final red blood cell container 776. During the transfer, the system 730 may also (optionally) add the additive solution required (if any) for the storage of the red blood cells (Step 892). It should be noted that, as the system 710 transfers the washed red blood cell/additive solution mixture, the mixture may pass through a leukoreduction filter 778 which, in turn, removes white blood cells (e.g., leukocytes) from the red blood cells.

After completing the first wash step and collecting the washed red blood cells, the system can then determine whether any red blood cells remain within the temporary red blood cell bag 774 (e.g., based upon the volume of whole blood processed, the hematocrit of the blood processed, and the volume of washed red blood cells collected, or on the weight of the bag) (Step 875). If there are no red blood cells remaining within the temporary red blood cell bag 774, the system 730 may transfer the additive solution required for storage (Step 982) and/or perform a rinse step to flush any red blood cells trapped in the filter 778 through the filter 778 and into the final RBC container 776 (Step 890). For example, the system 710 may energize the red blood cell pump 760, and transfer additional additive solution (e.g., 70 mL) from container 778 through line 779, line 770, and line 777, and through filter 778. As the additive solution passes through the filter 778, it flushes the trapped red blood cells out of the filter 778 and into the final red blood cell container 776 which, in turn, increases the red blood cell recovery. The procedure is then complete.

However, if red blood cells remain within the temporary red blood cell bag 774, the system 710 can then perform a second wash step (and subsequent wash steps, if necessary). For example, the system 710 can, depending on the weight of the temporary red cell bag 774, partially empty the bowl and can once again energize the red blood cell pump 760 and transfer the red blood cells within the temporary red blood cell bag 774 into the bowl 610 (Step 880). The system may then wash the red blood cells using the wash/additive solution in the additive solution bag 778 (Step 885). Once the second wash step is complete, the system 710 may then transfer the washed red blood cells to the final red blood cell bag 776. This wash cycle may be repeated until all red blood cells are washed and collected in the final red blood cell bag 776. The system 710 may then add the additive solution required for storage (Step 892) and rinse the filter 778 to flush the trapped red blood cells out of the filter 778 (Step 895).

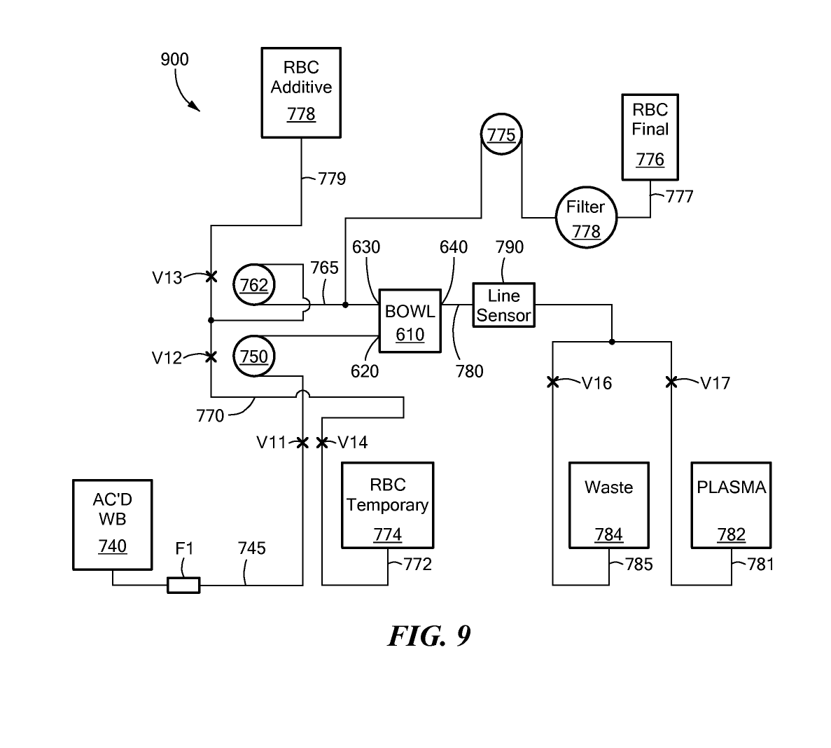

Although FIG. 7 shows a system 710 that utilizes only a single red blood cell pump 760 to draw both the pre-washed and the washed red blood cells out of the bowl 610, some embodiments may utilize more than one red blood cell pump. For example, as shown in FIG. 9, some embodiments can have (1) a red blood cell pump 762 that draws the pre-washed red blood cells from the bowl 610 and into the temporary red blood cell bag 774, and (2) a final red blood cell pump 775 that draws the washed (e.g., the final red blood cell product) from the bowl 610 and into the final red blood cell bag 776 (e.g., via line 777). It is important to note that in such embodiments, both red blood cell pumps 762/775 may be fluidly connected to the first blood component outlet 630 so that the red blood cells can be extracted from the bottom of the bowl (e.g., the extraction region 690).

It is also important to note that the method of processing whole blood using the system 900 shown in FIG. 9 is similar to that shown in FIG. 8. However, unlike the method shown in FIG. 8, after completing the wash steps (e.g., Step 885 in FIG. 8), the system 910 will energize the final red blood cell pump 775 (e.g., instead of the red blood cell pump 760 in FIG. 7 or the red blood cell pump 762 in FIG. 9) to transfer the washed red blood cells within the bowl 610 to the final red blood cell bag 776.

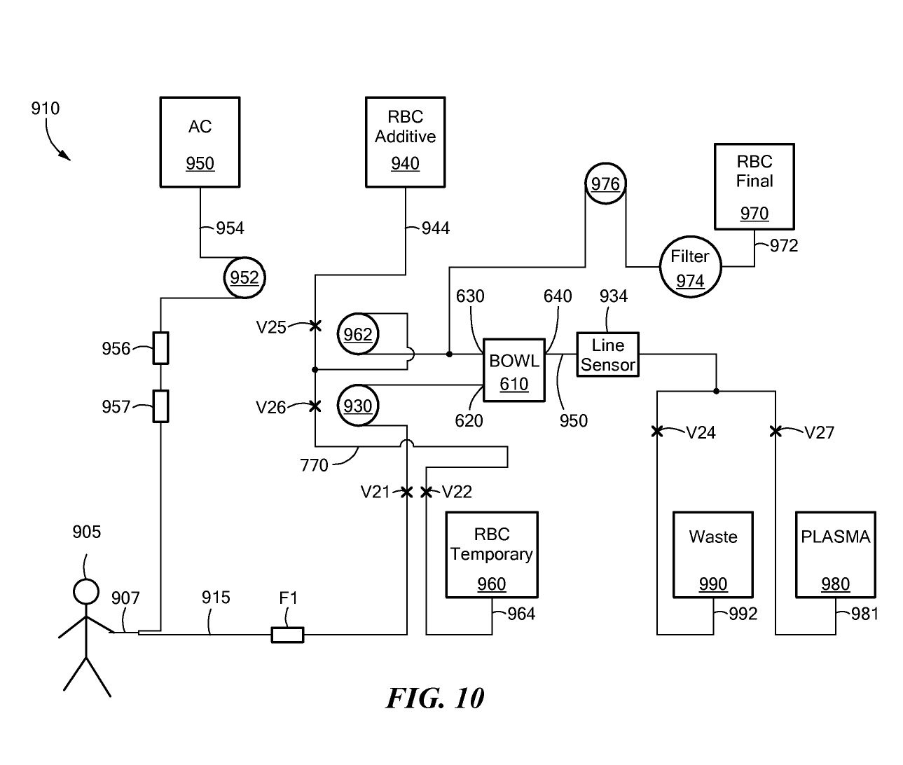

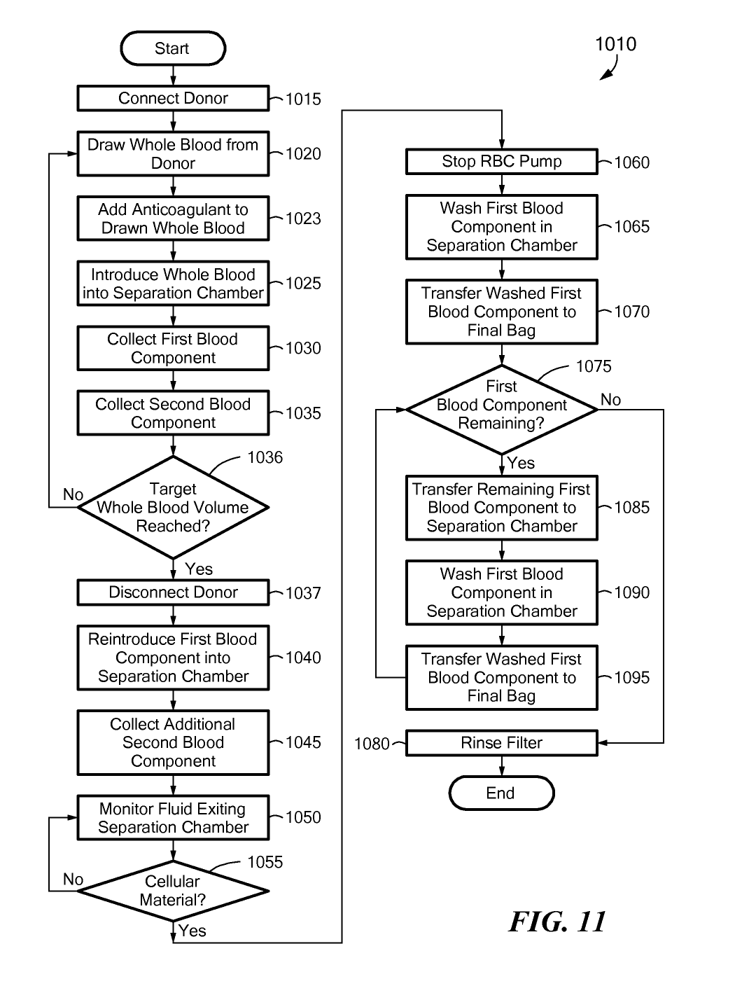

Although FIGS. 7, 8, and 9 show a system/procedure in which anticoagulated whole blood is drawn from a whole blood bag 740 and processed, other systems and methods can draw whole blood directly from the donor. FIGS. 10 and 11 schematically show one embodiment of a continuous flow system 910 for separating whole blood drawn from a donor, and a flow chart of an exemplary blood processing method 1010 using the system shown in FIG. 10, respectively. First, the user may connect the system 910 to the donor 905 using a venous access device 907 (Step 1015). Once the donor 905 is connected, the system 910 may then energize the draw pump 930 and begin drawing whole blood from the donor (Step 1020). As the whole blood flows through line 915 and V21, the system can also energize the anticoagulant pump 952, draw anticoagulant from an anticoagulant container 950 through an anticoagulant line 954, and begin mixing the anticoagulant with the whole blood being drawn from the donor (Step 1023). In some embodiments, the system 910 may have a filter 956 located on the anticoagulant line 954 to filter the anticoagulant before mixing with the whole blood and/or a blood filter F1 to filter the drawn whole blood. Additionally or alternatively, the anticoagulant line 954 may have an air detector to detect the presence of air bubble within the line 954.

The system 910 may then introduce the anticoagulated whole blood into the bowl 610 (e.g., via the inlet 610 and the inlet tube 625 and into the introduction region 667) (Step 1025), where the blood is separated into its individual components (e.g., red blood cells and plasma). Once the anticoagulated whole blood begins to separate, the system 910 may then begin collecting the red blood cells and plasma accumulating within the bowl 610. For example, in a manner similar to that described above for FIGS. 7-9, the system 910 may energize a red blood cell pump 962 and may begin extracting the red blood cells out of the bowl 610 (e.g., via the first blood component extraction tube 635 and the first blood component outlet 630), through lines 964, and into a temporary red blood cell storage container 960 (Step 830).

Simultaneously, as additional whole blood is introduced into the bowl 610, the whole blood displaces the plasma within the bowl 610 (e.g., in the separation region 665) and forces the plasma out of the second blood component outlet 640, through line 950, valve V27, and line 981 and into a plasma bag 980 where it is collected (Step 1035). The system 910 may continue the drawing, separation, and collecting processes (e.g., Steps 1020, 1030, and 1035) until a target volume of whole blood has been processed (Step 1036). Once the target volume of whole blood is reached, the system 910 may stop the draw pump 930, and the user/technician can disconnect the donor 905 (Step 1037).

The system 910 can then spin the centrifuge at a faster rate and transfer a portion of the red blood cells collected within the temporary red blood cell container 960 to the bowl 610 (Step 1040) using the red blood cell pump 962 (e.g., through line 964 and into the first blood component outlet 630). As the red blood cells are reintroduced into the bowl 610, the red blood cells will push the plasma remaining in the bowl 610 out through the second blood component outlet port 640, through line 950, valve V27, and line 981 and into the plasma container 980, where the additional plasma is collected (Step 1045). Like the embodiments shown in FIGS. 7 and 9, a line sensor 934 located on line 950 can monitor the fluid exiting the bowl 610 (Step 1050) and will detect when the fluid leaving the bowl 610 changes from plasma to a cellular material (e.g., platelets, red blood cells, etc.) (Step 1055). When the fluid exiting the bowl 610 changes from plasma to cellular material, the system 910 will stop reintroducing the red blood cells from the temporary red blood cell bag 960 (e.g., the system will stop pump 962) (Step 1060). At this point, the bowl 610 will be filled primarily with red blood cells.

As shown in FIG. 10, in addition to the temporary red blood cell bag 960, the system 910 may also have a final red blood cell bag 970 that is fluidly connected to the first blood component outlet 630 of the bowl 610 via line 972, and an additive solution bag 940 containing and an additive solution and fluidly connected to the inlet 620 of the bowl 610 via line 944. Once all of the plasma has been collected and the bowl 610 is full of red blood cells, the system 910 may then perform a wash step (optional) (Step 1065) to remove proteins from the red blood cells within the bowl. To that end, the system 910 (e.g., the controller) may energize the red blood cell pump 962 to draw additive solution from container 940 through line 944, and into the bowl 610 (e.g., through inlet port 620). As mentioned above, when additional additive solution enters the bowl 610 (e.g., when the bowl 610 is spinning), the additive solution/protein mixture will be displaced from the bowl 610 through the second blood component outlet port 640. The additive solution/protein mixture will then flow through line 950, valve V24, line 992 and into a waste container 990.

Once the wash step is completed, the system 910 may then transfer the washed red blood cells within the bowl 610 to the final RBC storage container 970 (Step 1070). For example, the system 910 may energize a final red blood cell pump 976 to draw the washed red blood cells from the bowl (via the inlet tube 625 and inlet 620), through line 972 and into the final red blood cell bag 970. In some embodiments, during the transfer, the system 910 may also (optionally and if needed) add the additive solution required for storage of the red blood cells. It should be noted that, as the system 910 transfers the red blood cell/additive solution mixture, the mixture may pass through a leukoreduction filter 974 which, in turn, removes white blood cells (e.g., leukocytes) from the red blood cells being collected in the final red blood cell bag 970.

After completing the first wash step and collecting the washed red blood cells, the system 910 can repeat the wash process for any red blood cells remaining within the temporary red blood cell bag 960. For example, like the systems shown in FIGS. 7 and 9, the system 910 can determine whether any red blood cells remain within the temporary red blood cell bag 960 (e.g., based upon the volume of whole blood processed, the hematocrit of the blood processed, and the volume of washed red blood cells collected, or on the weight of the temporary bag) (Step 1075). If red blood cells remain within the temporary red blood cell bag 960, the system 910 can, depending on the weight of the temporary red cell bag, partially empty the bowl and can once again energize the red blood cell pump 962 and transfer the red blood cells within the temporary red blood cell bag 960 into the bowl 610 (Step 1085). The system may then wash the red blood cells using the wash/additive solution in the additive solution bag 940 (Step 1090). Once the second wash step is complete, the system 910 may then transfer the washed red blood cells to the final red blood cell bag 970 (Step 1095). This wash cycle may be repeated until all red blood cells are washed and collected in the final red blood cell bag 960.

After completing any additional washing steps (or if there are no red blood cells remain within the temporary red blood cell bag 960 after the first was step), the system 910 may rinse the filter 974 on the line 972 leading to the final red blood cell bag 970 to force any red blood cells trapped in the filter 974 into the final RBC container 970 (Step 1080). For example, the system 910 may energize the red blood cell pump 976, and transfer additional additive solution (e.g., 70 mL) from container 940 through line 944 into the bowl 610. Next, the bowl will be emptied through the first component outlet port 630 and through line 972, and through the filter 974. As the additive solution passes through the filter 974, it flushes the trapped red blood cells out of the filter 974 and into the final red blood cell container 970 which, in turn, increases the red blood cell recovery. It is important to note that the plasma entering the plasma container 980 may later be filtered through a filter 982 and stored in filtered plasma container 984.

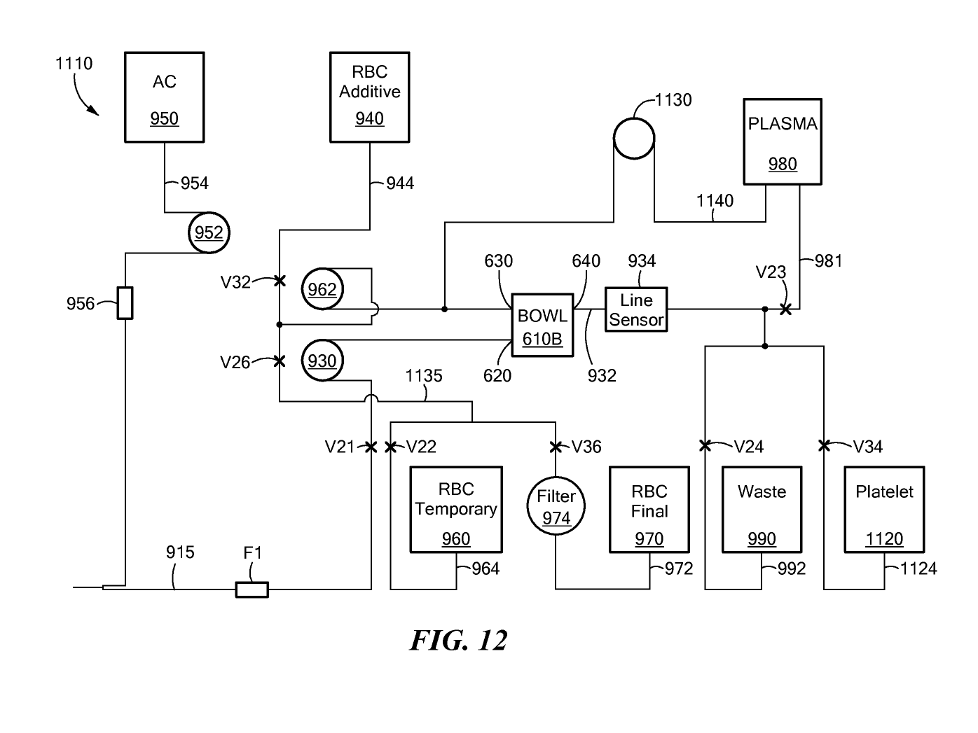

In addition to collecting red blood cells and plasma, some embodiments may also collect a third component (e.g., platelets). To that end, as shown in FIG. 12, in addition to the plasma bag 980, temporary and final red blood cell bags 960/970, and the waste bag 990, some embodiments (e.g., system 1110) can also include a platelet bag 1120 that is fluidly connected to the bowl 610B via lines 932 and 1124. Additionally, some embodiments seeking to collect the third blood component may also use the continuous flow centrifuge bowl 610B shown in FIG. 6B (e.g., as opposed to the continuous flow centrifuge bowl 610 shown in FIG. 6A) so that the whole blood is separated into red blood cells, plasma, and platelets. As discussed in greater detail below, after collecting the red blood cells in the temporary red blood cell bag 960 and the plasma within the plasma bag 980, the system 1110 can collect platelets within the platelet bag 1120.

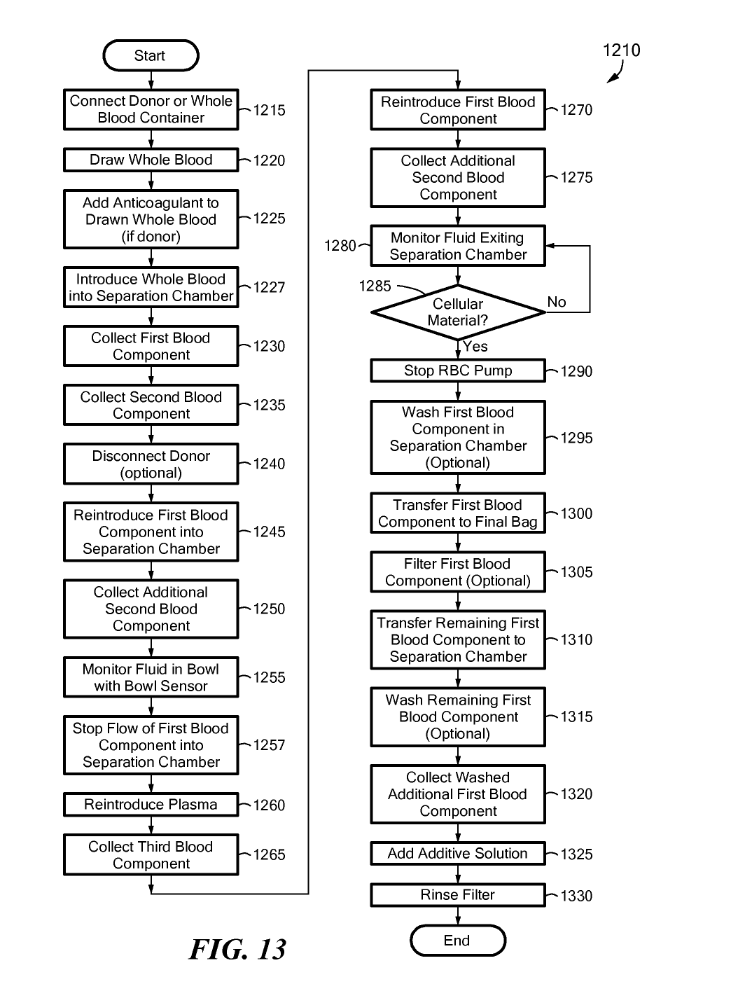

FIG. 13 shows a flow chart of an exemplary blood processing method 1210 using the system shown in FIG. 12. In the method, the user/technician may first connect the source of whole blood (e.g., a bag of anticoagulated whole blood or a donor) to the system 1110 (Step 1215), and may energize the draw pump 930 to begin drawing whole blood from the source, through line 915, blood filter F1, and valve V21 and into the bowl 610B via the inlet 620 (Step 1220). If the source is a donor, the system 1110 may also energize the anticoagulant pump 952 to draw anticoagulant from the anticoagulant bag 950 (through line 954) so that it mixes with the whole blood prior to entering the bowl 610B (Step 1225). If the source of whole blood is a bag of anticoagulated whole blood, the system 1110 may not need to add additional anticoagulant and, therefore, may skip this step.

Once the anticoagulated whole blood is introduced into the bowl 610B (Step 1227), the system may then begin collecting red blood cells (Step 1230) and plasma (1235) in a manner similar to that described above. For example, to collect the red blood cells, the system 1110 may energize the red blood cell pump 962 and begin drawing the red blood cells from the bowl via the first blood component outlet 630. Additionally, as additional whole blood enters the bowl 610B, the plasma will be forced out of the bowl and into line 932. The plasma may then flow through line 932, valve V23, line 981 and into plasma bag 980, where it is collected. If the source of whole blood is a donor, the user/technician can disconnect the donor once the target volume of whole blood is processed (Step 1240).

The system 1110 may then return some of the red blood cells collected within the temporary red blood cell bag 960 back to the bowl 610B (Step 1245), and may collect the additional plasma that is forced out of the bowl 610B as the red blood cells are introduced (Step 1250). It is important to note that, unlike the embodiments discussed above, where red blood cells are reintroduced until cellular material is detected at the line sensor 934, the process is different for the embodiment shown in FIGS. 12 and 13. Instead, the system 1110 monitors the reintroduction of red blood cells using the optical sensor 696 located on the bowl 610B (Step 1255) to determine when the bowl 610B is nearly full of red blood cells, but the platelets remain in the bowl 610B.

Once the optical sensor 696 detects that the bowl 610B is nearly full of red blood cells, the system 1110 can stop the red blood cell pump 962 (Step 1257), and energize a pump 1130 that is connected to the plasma container 980 to draw plasma from the plasma container 980. As the pump 1130 draws plasma out of the plasma container 980, the plasma is recirculated to/reintroduced into the bowl 610B via line 1140 (Step 1260) to perform an elutriation process to extract the platelets from the bowl 610B. For example, during the elutriation process, the system 1110 can gradually increase the flow rate of the pump 1130 to increase the flow rate of the plasma entering the bowl 610B. As the flow rate is increased, the drag force created by the plasma will eventually overcome the centrifugal force caused by the rotation of the bowl 610B, and the platelets will be carried away behind the plasma. As the fluid exiting the bowl 610B passes through the line sensor 934, the line sensor 934 will detect the change from plasma to platelets, and the system 1110 (or the operator if it is a manually operated system) will close valve V23 and open valve V34 so that the platelets flow through lines 932 and 1124 and into the platelet container 1120 (Step 1265).

After collecting the platelets, the system 1110 may then energize the red blood cell pump 962 and reintroduce additional red blood cells into the bowl 610B (e.g., through line 964 and the first blood component outlet 630) (Step 1270). During this time, the system 1110 can increase the speed of the bowl 610B to improve the separation within the bowl 610B and separate additional plasma (and other blood components) from the red blood cells entering the bowl 610B. As additional red blood cells enter the bowl 610B, the additional plasma is forced out of the bowl 610B, and the system can collect the additional plasma in the plasma container 980 (e.g., through the second blood component outlet 640, line 932, and line 981) (Step 1275). Additionally, as the plasma is exiting the bowl 610B, the line sensor 934 can monitor the fluid exiting the bowl 610B (Step 1280), and determine when the fluid changes to cellular material (Step 1285).

Once the line sensor 934 detects that the fluid exiting the bowl 610B is cellular material, the system 1110 can stop the red blood cell pump 962 (Step 1290) to stop the flow of red blood cells back into the bowl 610B. Although not necessary, the system 1110 can then optionally wash the red blood cells with additive solution (e.g., in a manner similar to that described above) (Step 1295).The system 1110 may then stop the bowl 610B and transfer the red blood cells within the bowl 610B to the final red blood cell bag 970. For example, the system 1110 can, once again, energize the red blood cell pump 962 to draw the red blood cells out of the bowl via the first blood component outlet 630. The red blood cells can then flow through lines 1135 and 972 and into the final red blood cell bag 970. As the red blood cells are transferred to the final red blood cell bag 970, the red blood cells may pass through the leukoreduction filter 974 which, in turn, removes white blood cells (e.g., leukocytes) from the red blood cells being collected in the final red blood cell bag 970. (Step 1305).

If additional red blood cells remain within the temporary first blood component bag 960, the system 1110 may transfer the remaining first blood component to the bowl 610B (Step 1310), wash the remaining red blood cells (Step 1315) (e.g., by repeating the wash process described above), and collect the additional washed first blood component from the bowl 610B (Step 1320) (e.g., by energizing the red blood cell pump 962 to draw them out of the bowl 610B). It is important to note that as the red blood cells are being drawn from the bowl 610B, the system 1110 can add additive solution to the red blood cells (Step 1325). For example, the system can energize pump 962 and draw the additive solution from the additive container 940 and through additive solution line 944 which connects with line 1135 (and, therefore line 972). The system 1110 may then rinse the filter 974 on the line 972 leading to the final red blood cell bag 970 to force any red blood cells trapped in the filter 974 into the final RBC container 970 (Step 1330).

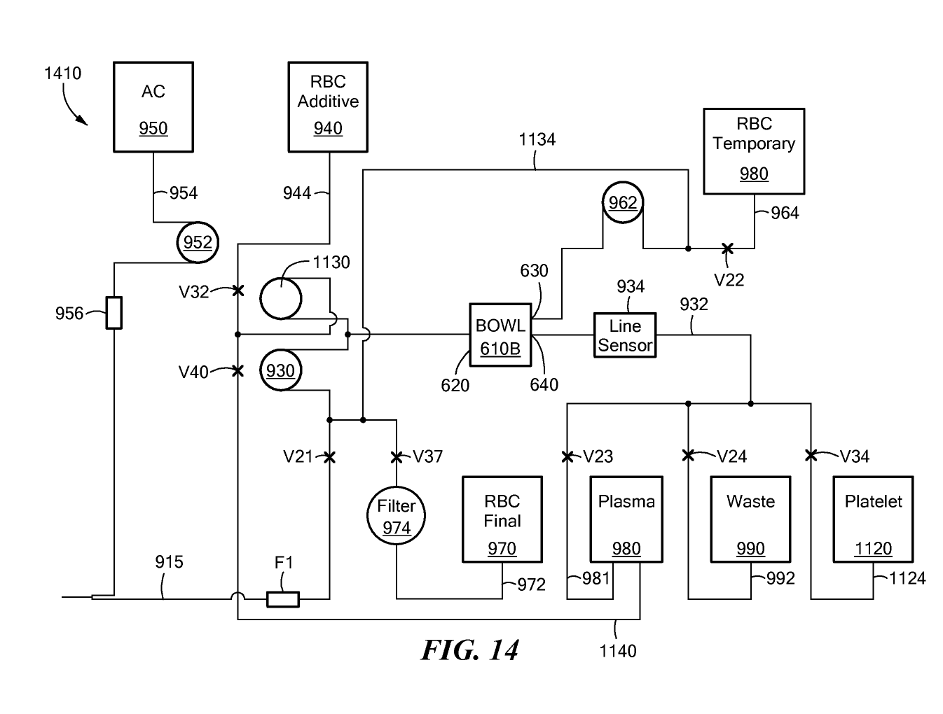

As shown in FIG. 14, some embodiments of the continuous flow three component systems (and the continuous flow two component systems) can have a different configuration that slightly changes the wash and additive solution addition processes. For example, although some of the embodiments described above introduce the additive solution into the first blood component outlet 630, other embodiments (e.g., system 1410 shown in FIG. 14) can introduce the additive solution into the inlet 620 of the bowl 610B when washing the red blood cells. To that end, the additive solution container 940 can be fluidly connected to the inlet 620 via the second blood component pump 1130. Additionally, during the wash cycle, the system 1410 can energize the second blood component pump 1130 to draw the additive solution through the additive solution line 944 and into the bowl 610B (through the inlet 620). To prevent the additive solution from entering the recirculation line 1140, the system 1410 can include a valve V40 that may be closed during the wash steps and when adding the additive solution to the red blood cells being transferred to the final red blood cell container 970.

It is also important to note that, in some embodiments, both the second blood component pump 1130 and the draw pump 930 can be energized when adding the additive solution necessary for storage of the red blood cells. For example, when collecting the washed red blood cells from the bowl 610B, the system 1410 can energize both the second blood component pump 1130 and the draw pump 930, and operate both pumps at the same speed in order to draw the additive solution from the additive solution container 940, and through additive solution line 944. The additive solution can then meet/mix with the red blood cells at the junction of lines 1134 and 972. Furthermore, in a similar manner, the system 1140 can utilize both the second blood component pump 1130 and the draw pump 930 to rinse the filter 974 after collecting the red blood cells within the final red blood cell container 970.

The embodiments of the invention described above are intended to be merely exemplary; numerous variations and modifications will be apparent to those skilled in the art. All such variations and modifications are intended to be within the scope of the present invention as defined in any appended claims.

1. An apparatus for separating whole blood comprising:
an access device through which whole blood is drawn from a source;
a continuous flow separation device in which the drawn whole blood is separated into a first blood component and a second blood component, the continuous flow separation device having an inlet for introducing whole blood into the continuous flow separation device, a first blood component outlet for withdrawing a first blood component, and a second blood component outlet for withdrawing a second blood component;
a draw line fluidly connecting the access device and the continuous flow separation device;
a draw pump that draws whole blood from the source through the access device and draw line and into the continuous flow separation device;
a first blood component storage container fluidly connected to the first blood component outlet;
a first blood component pump configured to draw the first blood component from the continuous flow separation device and into the first blood component storage container;
a final first blood component storage container fluidly connected to the first blood component outlet;
a second blood component storage container fluidly connected to the second blood component outlet; and
a controller for controlling fluid flow through the apparatus, the controller controlling operation of the draw pump and monitoring a total volume of whole blood drawn from the source, the controller configured to stop the draw pump when the target whole blood volume is withdrawn.

2-25. (canceled)

26. A method for separating whole blood comprising:
drawing whole blood from a source;
introducing, using a draw pump, the whole blood into a continuous flow separation device having an inlet, a first blood component outlet, and a second blood component outlet, the whole blood being introduced into the continuous flow separation device through the inlet;
separating in the separation chamber the whole blood into a first blood component and a second blood component, while whole blood is being drawn from the source, the whole blood displacing the second blood component from the continuous flow separation device via the second blood component outlet as additional whole blood is introduced;
extracting the first blood component from the separation chamber through the first blood component outlet;
collecting the extracted first blood component within a first blood component storage container;
collecting the displaced second blood component within a second blood component storage container while extracting the first blood component;
monitoring a total volume of whole blood drawn from the source to determine when a target whole blood volume is drawn; and
stopping, using a controller, the draw of whole blood from the source when the target whole blood volume is reached.

27-41. (canceled)

42. An apparatus for separating whole blood comprising:
an access device through which whole blood is drawn from a source;
a continuous flow separation device in which the drawn whole blood is separated into a first blood component, a second blood component, and a third blood component, the continuous flow separation device having an inlet for introducing whole blood into the continuous flow separation device, a first blood component outlet for withdrawing the first blood component, and a second blood component outlet for withdrawing the second blood component;
a draw line fluidly connecting the access device and the continuous flow separation device;
a draw pump configured to draw whole blood from the source through the access device and draw line and into the continuous flow separation device;
a first blood component storage container fluidly connected to the first blood component outlet;
a first blood component pump configured to draw the first blood component from the continuous flow separation device and into the first blood component storage container;
a final first blood component storage container fluidly connected to the first blood component outlet
a second blood component storage container fluidly connected to the second blood component outlet;
a second blood component pump fluidly connected to the second blood component container and the continuous flow separation device and configured to recirculate the second blood component collected within the second blood component container to the continuous flow separation device;
a third blood component storage container fluidly connected to the second blood component outlet; and
a controller for controlling fluid flow through the apparatus, the controller controlling operation of the draw pump and monitoring a total volume of whole blood drawn from the source, the controller configured to stop the draw pump when the target whole blood volume is withdrawn.

43-67. (canceled)

68. A method for separating whole blood comprising:
drawing whole blood from a source;
introducing, using a draw pump, anticoagulated whole blood into a continuous flow separation device having an inlet, a first blood component outlet, and a second blood component outlet, the whole blood being introduced into the continuous flow separation device through the inlet;
separating, in the continuous flow separation chamber, the whole blood into a first blood component, a second blood component, and a third blood component while blood is being drawn from the source;
extracting the first blood component from the continuous flow separation chamber through the first blood component outlet;
collecting the extracted first blood component within a first blood component storage container;
collecting the second blood component within a second blood component storage container while extracting the first blood component;
monitoring a total volume of whole blood drawn from the source to determine when a target whole blood volume is drawn;
stopping, using a controller, the draw of whole blood from the source when the target whole blood volume is reached;
recirculating at least a portion of the collected second blood component into the continuous flow separation device to displace the third blood component from the bowl; and
collecting the third blood component in a third blood component storage container.

69-82. (canceled)
